(19) United States
(12) Plant Patent Application Publication
(10) Pub. No.: US 2002/0002724 P1
(43) Pub. Date: Jan. 3, 2002

(54) Gladiolus plant named 'Golden Goddess'

(76) Inventor: John O. Zipperer III, Ft. Myers, FL (US)

Correspondence Address: Laura G. Barrow, Esq., P.O. Box 215, Estero, FL, 33928-0215, US

(21) Appl. No.: 09/877,574

(22) Filed: Jun. 8, 2001

(30) Foreign Application Priority Data
Jun. 9, 2000 (EP) 2000/0890

(51) Int. Cl. A01H 5/00

(52) U.S. Cl. PLT/301

(57) ABSTRACT

A new and distinct gladiolus variety, designated ‘Golden Goddess’, is shown and described. Compared to the ‘Jester Gold’ variety, the ‘Golden Goddess’ variety is 10-15 cm taller and has a larger bloom diameter (about 2-3 cm larger).

The present invention comprises a new and distinct variety of a Gladiolus plant referred to by the variety name ‘Golden Goddess.’

### BRIEF DESCRIPTION OF THE PHOTOGRAPHS

FIG. 1 is a photograph of a ‘Golden Goddess’ variety plant in bloom.

### ASEXUAL REPRODUCTION

The new variety was originated by the Applicant in a controlled proprietary breeding program in Ft. Myers, Fla. wherein selected gladiolus varieties were crossed. (The provisional breeder's designation “3-10” was used in a related application for a Community plant variety right filed earlier in the European Union Community Plant Variety Office). The female parent was a salmon pink gladiolus variety named ‘Jennie Lee,’ an unpatented released variety (by Jennie Lee Zipperer) characterized in part by a long stem, long head, and high floret count (i.e. 18-20 florets). The male parent was a rich yellow gladiolus variety named ‘Goldfield,’ characterized in part by having a good stem length, 15-16 florets, and a large susceptibility to Fusarium, Curvalaria, and Bottytis species. The seeds were planted in Ft. Myers, Fla., and the selection of ‘Golden Goddess’ variety was made in Spring 1988. Asexual reproduction of the ‘Golden Goddess’ variety was achieved by collecting cormels from the first corm. All subsequent asexual reproductions of the ‘Golden Goddess’ variety remain true to the original variety type.

### BOTANICAL DESCRIPTION

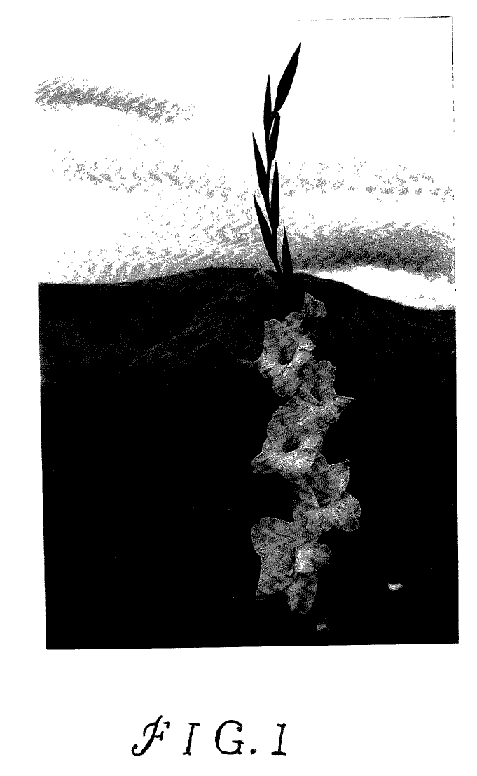

The accompanying color photograph (FIG. 1) shows the inflorescence and various stages of blooming of the ‘Golden Goddess’ variety plant.

The bud size of the ‘Golden Goddess’ variety is about 8 cm in length. The flowers of the variety each consist of a total of six petals. Specifically, each bloom consists of four large petals oriented on the top part of the bloom and a medium-size petal and a small-size petal oriented on the lower part of the bloom.

The color of the corolla bloom is purple deep within the throat of the bloom and a uniform yellow color extending throughout the rest of the bloom, with one of the two petals on the lower part of the bloom exhibiting a brighter yellow color compared to the five other petals. The diameter of the entire bloom is about 11 cm when using a #2 size corm.

The spike of the plant is about 130 cm and comprises 16 florets when grown from #2 size corms (1.25 to 1.50 inches) in Ecuador. It should be noted, however, that various factors will affect spike length, including temperature (larger spikes occur in cooler weather), irrigation, light intensity, fertilization, soil type (larger spikes occur in heavy soils versus sandy soil), and bulb size (larger bulbs result in larger spikes). The pistils of the flower are pale yellow, and the stamens are purple.

The leaf color of the ‘Golden Goddess’ variety is green. The leaf size is about 90 cm (length) by 3.5 cm (width).

### GENERAL OBSERVATIONS

The ‘Golden Goddess’ variety takes about 98 days to harvest in Ecuador. The variety can maintain six flowers in open bloom simultaneously in a vase of water after harvest from a tight cut stem. Compared to the gladiolus variety ‘Advance,’ the ‘Golden Goddess’ variety blooms a week later. Compared to the gladiolus variety ‘Jester Gold,’ the ‘Golden Goddess’ variety has a 2-3 cm larger bloom diameter and is 10-15 cm taller. Compared to the ‘Yellow Crown’ parent variety, the ‘Golden Goddess’ variety is a richer yellow and exhibits a greater tolerance to heat.

The ‘Golden Goddess’ variety has a good resistance to attack by Curvalaria fungi species but is similar to other yellow gladiolus varieties in its susceptibility to Botrytis. The variety grows well year round during long day lengths (i.e. 12-16 hours). The variety also shows a good resistance to burning in hot weather.

I claim:

1. A new and distinct gladiolus variety ‘Golden Goddess’, as shown and described herein.
